(19) United States
(12) Patent Application Publication
(10) Pub. No.: US 2024/0366831 A1
(43) Pub. Date: Nov. 7, 2024

(54) POROUS TITANIUM ALLOY STRUCTURE WITH A SILVER COATING HAVING OSTEOCOMPATIBILITY AND ANTIMICROBIAL PROPERTIES

(71) Applicant: WALDEMAR LINK GMBH & CO. KG, Hamburg (DE)

(72) Inventors: Helmut D. LINK, Hamburg (DE); Richard CSASZAR, Bad Segeberg (DE)

(73) Assignee: WALDEMAR LINK GMBH & CO. KG, Hamburg (DE)

(21) Appl. No.: 18/690,286

(22) PCT Filed: Sep. 30, 2022
(86) PCT No.: PCT/EP2022/077250

§ 371 Date: Mar. 8, 2024

Related U.S. Application Data
(60) Provisional application No. 63/250,882, filed on Sep. 30, 2021.

(51) Int. Cl. A61L 27/06; A61L 27/30; A61L 27/56

(52) U.S. Cl. CPC A61L 27/06; A61L 27/306; A61L 27/56; A61L 2300/104; A61L 2420/04

(57) ABSTRACT

Devices and methods that include a structure configured to contact a portion of a mammal's bone; and a coating on at least a portion of the structure, wherein the coating comprises a titanium coating component, a silver coating component, and a nitrogen coating component.

This application claims the benefit of U.S. Provisional Patent Application No. 63/250,882, filed Sep. 30, 2021, the disclosure of which is herein incorporated by reference in its entirety.

### BACKGROUND

Bone and joint pathologies account for approximately 50% of chronic illnesses in Europe, and osteoarthritis is the leading cause of disability in elder patients. End-stage osteoarthritis is typically treated by total joint arthroplasty that is based on the use of metallic implants. Additionally, minimized inflammatory reactions that can trigger bone resorption, which can in turn lead to implant loosening, are desirable, together with minimized bacterial infection risk.

Even though these events are often considered separately, they are related. For instance, upon bacterial infection, it has been suggested that adherent bacteria endotoxins may inhibit initial implant osseointegration. Implant loosening can occur due to bone resorption around an implant, either triggered by mechanical overload, or, conversely, by stress shielding. Porous titanium surfaces mimicking trabecular bone have been developed to improve osseointegration while minimizing stress shielding effects and relatively complex structures with customized porosities, pore geometries, sizes and shapes can be manufactured by additive manufacturing (AM).

Improving osseointegration is often attempted by enhancing the contact stability through the design or by implant surface modification. Chemical and physical modifications of implant surface(s) aimed at increasing surface roughness are commonly used, resulting in higher micromechanical retention to improve primary and secondary implant stability. In addition to surface roughness, which is often relevant at the nano or micro level, the macroarchitecture of the implant is a factor for new bone growth. Bone growth stimulation has been demonstrated to be impacted by total porosity and pore sizes. However, the higher surface area inherent to porous structures also increases the risk for bacterial colonization, leading to implant infection, the second most common reason for failure of arthroplasty implants after aseptic loosening.

Hence, efforts have been made to equip implantable biomaterials with long-term antimicrobial properties. Due to the development of antibiotic resistance and the lack of long-term effects, drug-eluting strategies based on antibiotics are not optimal. Furthermore, surface topographical patterns on implants inhibiting bacterial adhesion have also gained interest, such as the use of nanotubules, nanopillars or nanocones.

Mechanisms to provide biomaterials with antimicrobial properties either rely on a bacteriostatic surface which passively inhibits bacterial adhesion and proliferation, or on bactericidal surfaces that deliver antimicrobial agents capable of killing bacteria. Metal ion coatings have used as antimicrobial strategies for orthopedic implants.

Porous metallic implants offer a possible platform to improve osseointegration and minimize stress shielding. However, strategies to reduce the risk of bacterial adhesion and colonization of such implants are to be balanced against their potentially negative impact on osseointegration.

### SUMMARY

In accordance with one or more embodiments, devices and methods are provided.

The disclosure includes a device comprising: a porous, titanium alloy structure; and a coating on at least a portion of the structure, wherein the titanium alloy structure comprises a titanium structural component, wherein the coating comprises a titanium coating component, a silver coating component, and a nitrogen coating component.

The disclosure includes a device comprising: a structure configured to contact a portion of a mammal's bone; and a coating on at least a portion of the structure, wherein the coating comprises a titanium coating component, a silver coating component, and a nitrogen coating component.

The disclosure includes a method of inhibiting bacterial adhesion on an implantable apparatus, the method comprising: inserting the implantable apparatus into a portion of a mammal, to contact a portion of the mammal's bone, wherein the implantable apparatus comprises any suitable structure capable of insertion into a portion of a mammal and a coating on at least a portion of the structure, wherein the coating comprises a titanium coating component, a silver coating component, and a nitrogen coating component.

These and other advantages of the disclosure will be apparent to those of ordinary skill in the art by reference to the following detailed description and the accompanying drawings.

### BRIEF DESCRIPTION OF THE DRAWINGS

The patent or application file contains at least one drawing executed in color. Copies of this patent or patent application publication with color drawings will be provided by the Office upon request and payment of the necessary fee.

The accompanying drawings, which are incorporated in and constitute a part of this specification, illustrate embodiments of the disclosure, and, together with the summary given above, and the detailed description of the embodiments below, serve as a further explanation and disclosure to explain and/or illustrate embodiments of the disclosure.

FIGS. 1A and 1B are magnified view of an embodiment of a structure of the present disclosure.

FIG. 1C is a magnified view of an embodiment of a coating of the present disclosure.

FIGS. 2A, 2C, and 2D are X-ray diffraction (XRD) diffractograms for PoroLink® (PL), TrabecuLink® (TL) and silver-coated TrabecuLink® (TLSN) samples depicting main crystalline phases (A), and energy dispersive spectroscopy (EDS) color element mapping illustrating the surface composition of the three samples; titanium: green, aluminum: blue, oxygen: orange, silver: cyan, and nitrogen: red; scale bar: 100 μm. FIG. 2B shows X-ray photoelectron spectroscopy (XPS) survey spectra for the three scaffolds. FIGS. 2C and 2D are XRD diffractograms for PoroLink® (PL), TrabecuLink® (TL) and silver-coated TrabecuLink® (TLSN) samples depicting deconvoluted Ag 3d orbital spectra (FIG. 2C), and showing the highest signal for metallic Ag (binding energy: 368.3 eV), Ag0 (367.4 eV), and Ag silver clusters below 4 nm (369.2 eV) (FIG. 2D).

FIG. 3A shows scanning electron microscope (SEM) images of the three samples PoroLink® (PL), TrabecuLink® (TL), and silver-coated TrabecuLink® (TLSN) showing the morphology and porous structure at low magnification (first, upper row), and in-lens detector images depicting higher magnification details of the surface (second, lower row).

FIG. 3B shows white-light interferometry (WLI) surface 3D mapping depicting the roughness variations between the samples. First, upper row of each column visualizing a larger scanned area of the samples (1.68×1.68 mm²), and second row of each column showing a detailed higher magnification scanned area of 167×167 μm².

FIG. 4A is a graphical illustration of antimicrobial effects of the three samples at different time points, specifically S. aureus adhesion on the three scaffolds after 2 h of incubation.

FIG. 4B shows SEM images depicting adhered bacteria after 2 h on PoroLink® (PL), TrabecuLink® (TL), silver-coated TrabecuLink® (TLSN), scale bar: 5 μm (left column), and 1 μm (right column).

FIG. 4C is a graphical illustration of colony forming units (CFU) counts after biofilm formation by S. aureus for 72 h.

FIG. 4D is a graphical illustration of CFU counts after biofilm formation by S. epidermidis for 72 h. Throughout these figures, * denotes significant differences compared to PL, p<0.05.

FIG. 5A shows graphical illustrations of cell adhesion, proliferation and differentiation of SaOs-2 cells assessed by lactate dehydrogenase (LDH) and alkaline phosphatase (ALP) after 1, 7, and 14 days.

FIG. 5B shows immunostaining images depicting cells morphology seeded on the scaffolds PoroLink® (PL), TrabecuLink® (TL), silver-coated TrabecuLink® (TLSN) and control samples comprising glass coverslips (Ctrl) after 1 and 14 days of cell culture. a clear cytoplasm and nuclei disruption was observed on TLSN samples after 14 days; cell nuclei (blue) stained with 4′,6-diamidino-2-phenylindole (DAPI), and cell cytosol (green) stained with Carboxyfluorescein diacetate (CFDA); scale bar: 500 μm.

FIG. 6A shows graphical illustrations of cell adhesion, proliferation and differentiation of hOB cell assessed by LDH and ALP production after 1, 7, and 14 days. FIG. 5B shows immunostaining images depicting morphology of cells seeded on the scaffolds PoroLink® (PL), TrabecuLink® (TL), silver-coated TrabecuLink® (TLSN), and control samples comprising glass coverslips (Ctrl) after 1 and 28 days of cell culture.

In FIG. 6B, cell nuclei (blue) stained with DAPI, and cell cytosol (green) stained with CFDA, (scale bar: 500 μm), are shown.

FIG. 7 shows SEM images of human osteoblast (OB) cultured on PoroLink® (PL), TrabecuLink® (TL), silver-coated TrabecuLink® (TLSN) scaffolds, and control samples comprising glass coverslips (Ctrl) at 1, 7, 14 and 28 days, illustrating the increasing cell coverage of all samples over time at low magnification (white scale bar: 50 μm), and high magnification (black scale bar: 500 μm).

FIG. 8 shows graphical illustrations of human OB expression of osteogenic related genes Runt-related transcription factor 2 (RUNX2), collagen type I alpha 1 (COL1A1) and alpha 2 (COL1A2), alkaline phosphatase (ALP), and osteocalcin (OCN) at 3, 7, 14 and 28 days of cell culture on PoroLink® (PL), TrabecuLink® (TL), silver-coated TrabecuLink® (TLSN) scaffolds, and quantification of mineralization by alizarin red (AR) staining after 14 and 28 days.

FIG. 9 shows immunostained images of human osteoblasts (hOB) hOB cultured on PoroLink® (PL), TrabecuLink® (TL), silver-coated TrabecuLink® (TLSN) scaffolds, and control samples comprising glass coverslips (Ctrl) after 14 and 28 days showing cell nuclei (blue, DAPI), cytosol (green, CFDA), osteocalcin (red, OCN) and merged channels (scale bar: 100 μm).

FIG. 10A is a graphical illustration of silver release in cell culture media from silver-coated TrabecuLink® (TLSN) samples over 28 days depicting both punctual release and cumulative release measure by ICP.

FIG. 10B shows optical microscope images of time-lapse hOB cultures in presence of silver nitrate, AgNO₃, supplemented to cell culture media over a period of 5 days (t0=120 h) and final time-lapse (tf=140 h); scale bar: 250 μm.

FIGS. 11A, 11B, 11C, and 11D are black and white views of FIGS. 2A, 2B, 2C, 2D, respectively.

FIGS. 12A and 12B are black and white views of FIGS. 3A and 3B, respectively.

FIGS. 13A and 13B are black and white views of FIGS. 5A and 5B, respectively.

FIGS. 14A and 14B are black and white views of FIGS. 6A and 6B, respectively.

FIG. 15 is a black and white view of FIG. 8.

FIG. 16 is a black and white view of FIG. 9.

In the following description, it is to be understood that whenever reference is made to one of the figures in color, reference is also made in parallel to the corresponding black and white view.

### DETAILED DESCRIPTION

To facilitate the understanding of this disclosure a number of terms of in quotation marks are defined below. It is noted that the drawings of the present application are provided for illustrative purposes only and, as such, the drawings are not drawn to scale. It is also noted that like and corresponding elements are referred to by like reference numerals.

In the following description, numerous specific details are set forth, such as particular structures, components, materials, dimensions, processing steps and techniques, in order to provide an understanding of the various embodiments of the present application. However, it will be appreciated by one of ordinary skill in the art that various embodiments of the present application may be practiced without these specific details. In other instances, well-known structures or processing steps have not been described in detail in order to avoid obscuring the present application.

It will be understood that when an element as a layer, region or substrate is referred to as being “on” or “over” another element, it can be directly on the other element or intervening elements may also be present. In contrast, when an element is referred to as being “directly on” or “directly over” another element, there are no intervening elements present. It will also be understood that when element is referred to as being “beneath” or “under” another element, it can be directly beneath or under the other element, or intervening elements ay be present. In contrast, when an element is referred to as being “directly beneath” or “directly under” another element, there are no intervening elements present.

As used herein, the term “substantially” or “substantial”, is equally applicable when used in a negative connotation to refer to the complete or near complete lack of an action, characteristic, property, state, structure, item, or result. For example, a surface that is “substantially” flat would either be completely at, or so nearly flat that the effect would be the same as if it were completely flat.

As used herein, terms defined in the singular are intended to include those terms defined in the plural and vice versa.

As used in this specification and its appended claims, terms such as “a”, “an” and “the” are not intended to refer to only a singular entity, but include the general class of which a specific example may be used for illustration, unless the context dictates otherwise. The terminology herein is used to describe specific embodiments of the invention, but their usage does not delimit the invention, except as outlined in the claims.

Unless otherwise indicated, all numbers expressing quantities of ingredients, properties such as molecular weights, reaction conditions, and so forth as used in the specification and claims are to be understood as being modified in all instances by the term “about.” Accordingly, unless indicated to the contrary, the numerical parameters in the specification and claims are approximations that may vary depending upon the desired properties sought to be obtained by the present invention. At the very least, and without limiting the application of the doctrine of equivalents to the scope of the claims, each numerical parameter should at least be construed in light of the number of reported significant digits and by applying ordinary rounding techniques. Notwithstanding that the numerical ranges and parameters describing the broad scope of the invention are approximations, the numerical values in the specific examples are reported as precisely as possible. Any numerical value, however, inherently contains standard deviations that necessarily result from the errors found in the numerical value's testing measurements.

Thus, reference herein to any numerical range expressly includes each numerical value (including fractional numbers and whole numbers) encompassed by that range. To illustrate, reference herein to a range of “at least 50” or “at least about 50” includes whole numbers of 50, 51, 52, 53, 54, 55, 56, 57, 58, 59, 60, etc., and fractional numbers 50.1, 50.2 50.3, 50.4, 50.5, 50.6, 50.7, 50.8, 50.9, etc. In a further illustration, reference herein to a range of “less than 50” or “less than about 50” includes whole numbers 49, 48, 47, 46, 45, 44, 43, 42, 41, 40, etc., and fractional numbers 49.9, 49.8, 49.7, 49.6, 49.5, 49.4, 49.3, 49.2, 49.1, 49.0, etc. In yet another illustration, reference herein to a range of from “5 to 10” includes whole numbers of 5, 6, 7, 8, 9, and 10, and fractional numbers 5.1, 5.2, 5.3, 5.4, 5.5, 5.6, 5.7, 5.8, 5.9, etc.

In the discussion and claims herein, the term “about” indicates that the value listed may be somewhat altered, as long as the alteration does not result in nonconformance of the process or structure to the illustrated embodiment. For example, for some elements the term “about” can refer to a variation of ±0.1%, for other elements, the term “about” can refer to a variation of ±1% or ±10%, or any point therein.

As used herein, the term “agglomerate(s)” refers to a plurality of particles wherein the particles can be physically and/or chemically, irreversibly and/or reversibly, adhered together to form a discrete body of matter. As used herein, the term “adhered” means the particles are physically and/or chemically bound or bonded together. As used herein, the term “adhered” is not intended to be limiting as to the nature of the binding/linking between the particles.

As referred to herein, the terms silver-coated TrabecuLink® and/or TLSN refer to an embodiment of the present disclosure.

The coated, porous, titanium alloy structure of the present disclosure can form any portion of any device or element that can be implanted into a mammalian body and contact a bone of that mammal. For example and without limitation, the porous titanium alloy structure of the present disclosure can form any portion of any hip implant/replacement, pelvic implant/replacement, shoulder implant/replacement, knee implant/replacement, ankle implant/replacement, elbow implant/replacement, wrist implant/replacement, a revision prosthesis, jaw implants/replacements, dental implants/replacements, spinal implants/replacements, etc. In addition, the coating of the present disclosure can be on any portion of any surface of any device or element that can be implanted into a mammalian body and contact a bone of that mammal, including the examples listed above, as well as others.

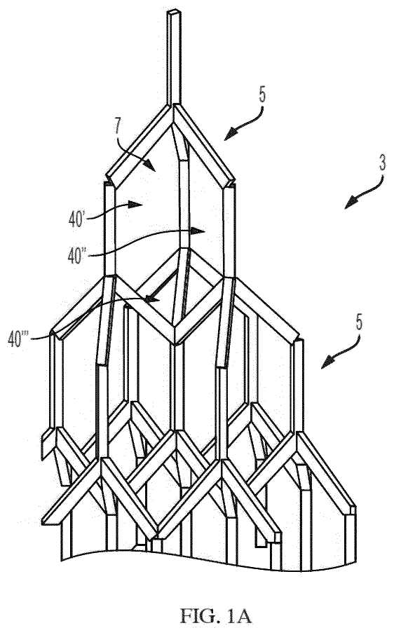

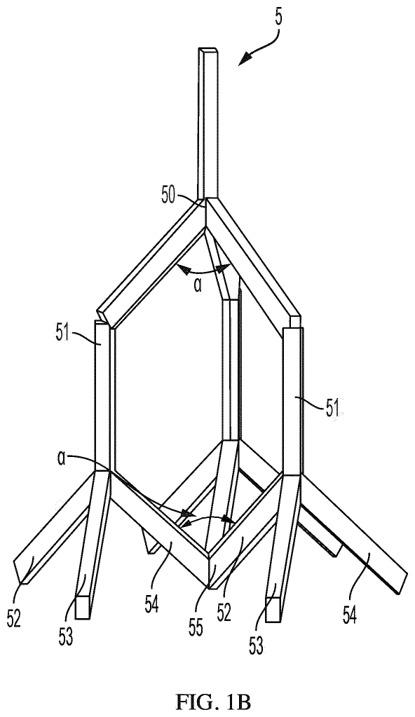

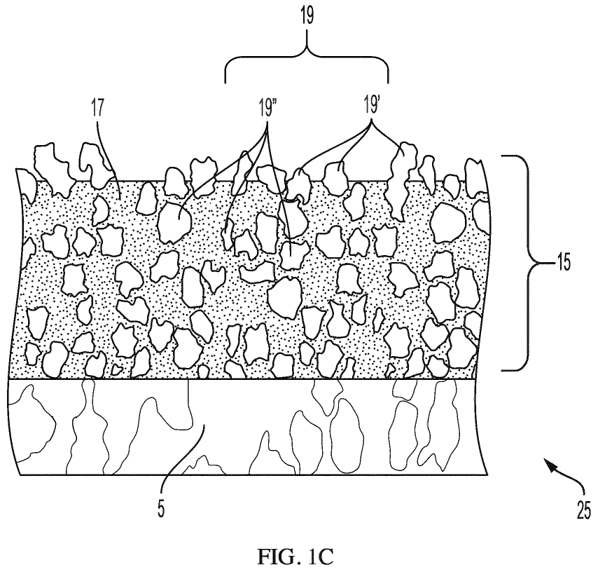

An embodiment of the present disclosure is shown in FIG. 1A. As can be seen in FIG. 1A, an array 3 of porous, titanium alloy structures 5 are illustrated. One arrangement of structures 5 within the array 3 is shown in FIG. 1A, and can be arranged in layers within the array 3, however, details of arrangement differ between the directions of view of the array 3, with the arrangement being modifiable as desired.

Additionally, FIG. 1A illustrates one example of the arrangement of the array 3 and the shape of the porous, titanium alloy structure 5, however, in other embodiments, any suitable array 3 wherein adjacent structures 5 contact other adjacent structures at at least one point can be used. Also in other embodiments, any shape of porous, titanium alloy structure 5 can be included such that each element of the structure 5 can have any cross-sectional shape, and that the elements of the structure 5 can form any size pores of any suitable shape, with each element being the same or different from adjacent elements.

Each structure 5 of the array 3 is porous, for example having pores 40′, 40″ and 40′″, among others. The size of each of the pores 40′, 40″ and 40′″ is individually adjustable, with each of the 40′, 40″ and 40′″ being the same sizes and/or shapes as compared to each other (unimodal) or some or all of the pores 40′, 40″ and 40′″ can be different sizes and/or shapes as compared to each other (multimodal). As an example, in some embodiments, one or more of pores 40′, 40″ and 40′″ can have a size between about 400 μm and 1,100 μm, or about 500 μm with an overall porosity of about 90% or more, about 80%, about 70%, about 65%, about 60%, about 55%, about 50%, about 40%, or about 30% or less.

Within each structure 5 there is a free space 7, which is interconnected with the internal free-space of neighboring structures 5. In this embodiment, each structure 5 of the array 3 can be formed through any suitable additive manufacturing (AM) and/or 3D-printing technology. For example, each structure 5 of the array 3 can be formed according to a suitable electron beam melting (EBM) process. EBM is an additive process for manufacturing and may produce solid or porous material. A powder of the desired material is provided in the desired granulometry. By the EBM process the powders of the desired material are deposited in successive layers at desired positions and in desired sequence (as defined in a preceding modelling step for the porous structure) and made to melt such as to form a coherent body.

In this embodiment, the material of the porous, titanium alloy structure 5 comprises a titanium structural component. The porous, titanium alloy structure 5 can further comprise at least one of an aluminum structural component and a vanadium structural component. In any embodiment, the relative concentrations of all elements can be adjusted as desired. One example of the material of the porous, titanium alloy structure 5 is Ti-6Al-4V.

A more magnified view of one structure 5 of the present disclosure is shown in FIG. 1B. Each of the structures 5 is formed by “basic” elements, wherein a basic element is shaped like, for example, a tetrapod, but in other embodiments any suitable shape can be formed. A tetrapod is a structure having four legs 51, 52, 53, 54 being connected at a center point 50, each of the legs 51, 52, 53, 54 pointing away from the center point 50 and spanning with their free ends, for example a tetrahedron, but in other embodiments different shapes can be formed.

The tetrahedron, or any other suitable shape, may be irregular or regular. Optionally an isosceles tetrahedron is formed wherein each of the legs 51, 52, 53, 54 would form the same angle to each of the other three legs. In the context of the present disclosure a standard orientation of the tetrapod shall be one of the legs 51, 52, 53, 54 pointing upwards (“top leg” 51) and the other three legs forming a stand (“base legs” 52, 53, 54), wherein the far ends of the three base legs 52, 53, 54 define a base plane.

As can be seen in FIG. 1B, an angle formed between the legs forming the 55 and the legs forming the 50 be smaller than other angles of the structure 5. In some embodiments, the angle α can be about 120° or less, or about 115° or less, or about 110° or less, or about 109° or less, or about 105° or less, or about 100° or less, or about 99° or less, or about 98° or less, or about 97° or less, or about 96° or less, or about 95° or less, or about 90° or less.

The porous, titanium alloy structure 5 of FIGS. 1A and 1B is shown without a coating, for illustrative purposes, a portion of a coating 15 is shown in FIG. 1C. The coating 15 can be on at least a portion of the porous, titanium alloy structure 5. The coating 15 can comprise a titanium coating component, a silver coating component, and a nitrogen coating component. The titanium coating component can be any titanium containing material, including titanium alloys. The silver coating component can be any silver containing material, including silver alloys. The nitrogen coating component can be any nitrogen containing material.

In some embodiments the silver coating component can comprise silver agglomerates 19. In some embodiments the titanium coating component and the nitrogen coating component comprise titanium nitride 17.

The coating 15 can be applied to any portion of the porous, titanium ally structure 5 using any suitable method, such as physical vapor deposition (PVD). During any suitable application process, such as PVD, the coating 15 can be applied in a substantially uniform film thickness (about 4.5 μm±3.5 μm, or about 4.5 μm±3 μm, or about 4.5 μm±2.5 μm, or about 4.5 μm±2 μm, or about 4.5 μm±1.5 μm, or about 4.5 μm±1 μm, or about 4.5 μm±0.5 μm, or about 3 μm to about 6 μm) and composition.

Upon application of the coating, in some embodiments a first portion 19′ of the silver agglomerates 19 can be partially within, and partially exposed from, the titanium nitride 17. In some embodiments a second portion 19′ of the silver agglomerates 19 are within the titanium nitride 17. However, in other embodiments, all, or substantially all, of the silver agglomerates 19 can be partially within, and partially exposed from the titanium nitride 17. Also, in other embodiments, all, or substantially all, of the silver agglomerates 19 can be within the titanium nitride 17.

The material of the coating 15 can be any suitable combination of titanium coating component, silver coating component and nitrogen coating component. For example, the coating 15 can comprise, as any endpoint of any range, about 20% at % titanium coating component, or less, about 25% at % titanium coating component, about 30% at % titanium coating component, about 33.2% at % titanium coating component, about 35% at % titanium coating component, about 40% at % titanium coating component, about 45% at % titanium coating component, about 50% at % titanium coating component, or more, with at % referring throughout this disclosure to atomic percentage. For example, the coating 15 can comprise, as any endpoint of any range, about 5% at % silver coating component, or less, about 6% at % silver coating component, about 7% at % silver coating component, about 8% at % silver coating component, about 9% at % silver coating component, about 10% at % silver coating component, about 11% at % silver coating component, about 12% at % silver coating component, about 13% at % silver coating component, about 14% at % silver coating component, about 15% at % silver coating component, or more. For example, the coating 15 can comprise, as any endpoint of any range, about 40% at % nitrogen coating component or less, about 45% at % nitrogen coating component, about 50% at % nitrogen coating component, about 55% at % nitrogen coating component, about 56.5% at % nitrogen coating component, about 60% at % nitrogen coating component, about 65% at % nitrogen coating component, about 70% at % nitrogen coating component, or more.

Although not illustrated, an optional Zirconium comprising layer or coating may be included on at least a portion of the porous, titanium alloy structure 5, between the porous, titanium alloy structure 5 and a portion of the coating 15.

The structure 5 and the coating 15 together form a device 25 of the present disclosure.

In other embodiments of the disclosure the coating 15 can be used in a method for inhibiting bacterial adhesion on an implantable apparatus, such as structure 5. This method includes the step of inserting, using any suitable method or technique, an implantable apparatus into a portion of a mammal, to contact a portion of that mammal's bone. The implantable apparatus can be any structure capable of insertion into a mammal, such device 25 and/or any implant or structure discussed in the paragraphs above or herein, that is at least partially coated with coating 15.

The ability of to inhibit bacterial adhesion on the implantable apparatus is discussed in the Examples section below, and illustrated in the figures of the present disclosure.

### EXAMPLES

In the following examples, a porous electron beam melted (EBM) Titanium (Ti) alloy scaffold with a physical vapor deposition (PVD)-applied silver coating, a scaffold of the present disclosure, was evaluated for its capacity to both promote osseointegration while also conferring antibacterial properties. As a comparison, two clinically used and available Ti alloys, one dense grit-blasted Ti sample, and one relatively similar EBM porous TI allow without a silver coating were used as controls.

The sample of the present disclosure and the relatively similar EBM porous structure both had pore sizes of about 500 nm-about 600 μm and about 5 μm to about 10 μm surface roughness, the silver-coated samples contained about 7% Ag, resulting in a cumulative Ag release of about 3.5 ppm up to about 28 days.

As noted below, silver reduced the adhesion of Staphylococcus (S.) aureus to porous samples and inhibited 72 h biofilm formation by S. epidermidis. Primary human osteoblast adhesion, proliferation and differentiation were not substantially impaired in the presence of silver, and expression of osteogenic genes as well as production of mineralized matrix were observed in scaffolds of the present disclosure.

The Examples below indicate that silver coating of porous titanium implants can achieve antimicrobial effects without substantially compromising osteocompatibility.

### 1. Materials and Methods

### 1.1 Samples Production

The three investigated samples all consisted of Ti6Al4V alloy as the base material but were differently manufactured, always resulting in discs of 12.5 mm diameter and a height of 2 mm. Dense PoroLink® (PL) samples were manufactured from Ti6Al4V extra low interstitials (ELI) bars, turned on a bench lathe and sliced to discs. Subsequently, the discs were deburred and grit-blasted with corundum to a target surface roughness of 10 μm. TrabecuLink® (TL) samples were additively manufactured from Ti6Al4V extra low interstitial (ELI) powder in an electron beam machine (Arcam EBM Q10 plus). A solid base of 1 mm followed by a 1 mm porous structure based on a trabecular structure were manufactured using a support structure to prevent distortion. The supports were then mechanically pinched off.

TrabecuLink® scaffolds of the present disclosure, specifically scaffolds referred to as (“TLSN”) in the present disclosure, were manufactured as TL samples and coated after EBM manufacturing. After printing, TL samples were attached in a fixture with two set screws centrally pinning the discs' front and back face. The fixtures were then mounted on a central rotary plate inside the physical vapor deposition (PVD) coating chamber (PVD, Voestalpine eifeler alpha 400P) with the trabecular surface facing the twelve titanium and silver targets (cathodes) placed in three rows on the left and right side of the chamber. Each level of samples was aligned with a row of targets. Opposing direction of rotation at different speeds of the central rotary plate and individual fixtures plus varying target engagement was applied in an attempt to achieve a substantially uniform film thickness (about 4.5 μm±3.5 μm, or about 4.5 μm±3 μm, or about 4.5 μm±2.5 μm, or about 4.5 μm±2 μm, or about 4.5 μm±1.5 μm, or about 4.5 μm±1 μm, or about 4.5 μm±0.5 μm) and composition. Before the deposition stage of the process, the specimen surface was glow-discharge cleaned utilizing hydrogen and argon. The deposition stage was conducted in a nitrogen atmosphere at temperatures up to 460° C. High current, low voltage electric arcs vaporized the target materials and the ionized vapor was accelerated and deposited on the substrate forming the coating.

The entire process lasted about 200 min of which about 100 min were the effective deposition duration. All samples were subjected to an ultrasonic and a steam cleaning. TL samples were additionally cleaned with modified alcohols beforehand to remove residual powder from the EBM process. Finally, all samples were sterilized by gamma irradiation with a dose between 25 and 50 kGy before use.

Although the deposition process was described in detail above, by use of PVD, the coating of the present disclosure can be effected with any suitable technology capable of coating a porous, titanium alloy structure with a coating comprising a titanium coating component, a silver coating component, and a nitrogen coating component.

### 1.2 Samples Characterization

The morphology of the samples was examined by high resolution field emission scanning electron microscopy (FE-SEM, Merlin Zeiss, Germany), and energy dispersive X-ray spectroscopy (EDS, Merlin Zeiss, Germany), at an operating voltage of 5 kV for surface imaging and 15 kV for their chemical composition at surface level. The phase composition was investigated through X-ray diffraction (XRD, D8 Advance, Bruker, Germany) using Bragg-Brentano geometry, equipped with Cu Kα anode, and operated at 40 kV and 40 mA. Data were collected from 20 to 80° over 2Θ range and compared to the experimental patterns of titanium (Ti, JCDPS 44-1294), titanium nitride (TIN, JCPDS 38-1420), silver (Ag, JCPDS 01-1164), and corundum/alumina (Al2O3, JCPDS 01-085-1337).

Quantitative elemental composition was investigated on the surfaces of the scaffolds by X-ray photoelectron spectroscopy (XPS, Physical Electronics Quantera II, USA, Inc.) equipped with Al monochromatic Kα beam, operated at 25 W and 15 kV. Survey spectra were acquired for each scaffold using a pass energy of 55 eV and 20 ms time per step, and corrected using the C 1s signal. The spectra were analyzed using CasaXPS software.

White light interferometry (WLI, Nex View Zygo, Ametek Inc., Weiterstadt, Germany) was used to investigate the surface roughness of the three scaffolds. Four samples of each type were analyzed with a scanned area 1.68×1.68 mm² using a 5× magnification, and four different sites from each single measurement were included. Similarly, additional measurements with a smaller scanned area of 167×167 μm² and a 50× magnification were performed to assess the roughness of the printed pillars in the porous samples, TrabecuLink® (TL) and silver-coated TrabecuLink® (TLSN). The asymmetry in height distribution (skewness, Rsk), and the sharpness/randomness of heights in the surfaces (kurtosis, Rku), together with the profile average height (Ra) were quantified.

### 1.3 Antimicrobial Effects

### 1.3.1 Bacterial Inoculation

Two bacterial strains derived from patients with periprosthetic joint infection (PJI) treated at Uppsala University Hospital were used for the microbiology studies, Staphylococcus aureus (AN-1400743) and Staphylococcus epidermidis (AN-1400573). Each strain was thawed using 1 μL loop, inoculated in a 3% horse blood agar plate, and incubated overnight at 37° C. Then, colonies were collected using a 1 μL loop, and diluted in 0.9% wt. sterile sodium chloride (NaCl, 1/50,000 dilution, Sigma Aldrich, Sweden). The bacterial suspension was the further diluted 1/10 in tryptic soy broth (TSB, Soybean-Casein Digest Medium, BD Bioscience, Sweden) and used for the following experiments discussed in this disclosure. Control samples comprising dense PoroLink® (PL) were incubated in TSB solution without bacteria to assess that the only bacteria counts corresponded to the inoculated strains.

### 1.3.2 Bacterial Adhesion

S. aureus bacterial adhesion on the three samples was investigated after 2 h of incubation under static conditions. 3 mL of the bacterial suspension in TSB were added to each sample and incubated at 37° C. for 2 h. Afterwards, samples were rinsed twice in phosphate buffered saline (PBS, Sigma Aldrich, Sweden), transferred to new vials, and the adhered bacteria were detached by an enzymatic treatment comprising 2 mg/mL dispase and 4 mg/mL collagenase in 0.9% wt. NaCl solution. 600 μL of the enzymatic solution was added to each sample, vortexed for about 30 s, and incubated at 37° C. for 2 h under agitation at 220 rpm. Then, the samples were further vortexed for 2 min, and the detached bacteria diluted and seeded on blood agar plates and incubated at 37° C. Colony forming unit (CFU) counting was performed after 24 h and expressed as CFU/mL divided by the contact area of the scaffold (about 1.23 cm² for PL, and about 2.68 cm² for porous TL and TLSN). Three technical replicas were used for each sample type, and four biological replicas were used. Adherent bacteria after 2 h of incubation were visualized by FE-SEM. One sample of each scaffold was incubated in the bacterial suspension, rinsed twice in phosphate-buffered saline (PBS, Gibco, Merck, KGaA, Darmstadt, Germany) and then fixed using 4% formaldehyde for 30 min at room temperature. Next, the samples were dehydrated in increasing ethanol series (10-30-50-70-90-96-99%), sputtered with a 5-8 nm gold-palladium layer and imaged by FE-SEM (FE-SEM, Merlin Zeiss, Germany).

### 1.3.3 Biofilm Formation

Biofilm formation on the three samples was investigated after 72 h using two strains, S. aureus and S. epidermidis. Similarly, 3 mL of each independent bacterial suspension in TSB were added to each sample and incubated at 37° C. under static conditions. After 72 h of incubation, samples were removed from the bacterial suspension, rinsed twice with PBS, and placed in new sterile vials. 600 μL of enzymatic solution comprising 2 mg/mL dispase and 4 mg/mL collagenase in sterile 0.9% wt. NaCl solution was added, as noted in 1.3.2 above, incubated at 37° C. for 2 h under agitation at 220 rpm, and vortexed for 2 min. The detached bacteria were diluted and seeded on blood agar plates. After 24 h of incubation at 37° C., CFU were counted, and expressed as CFU/mL divided by the contact area of the scaffolds. Three technical replicas were used for each sample type, and six biological replicas were used.

### 1.4 Cell Studies

### 1.4.1 Osteoblast Proliferation and Differentiation

Pre-osteoblastic osteosarcoma cell line (SaOs-2, ECACC, Sigma Aldrich, United Kingdom) was used to study cell viability on all scaffolds. The scaffolds were incubated in 1 mL complete media comprising Dubelcco's modified Eagle's medium low glucose (DMEM, Merck, KGaA, Darmstadt, Germany) supplemented with 10% fetal bovine serum (FBS, Merck, KGaA, Darmstadt, Germany), 1% penicillin/streptomycin, and 0.5% amphotericin overnight. The next day, 10⁶ cells were seeded in a 50 μL droplet, and the scaffolds were left for 1 h in the incubator (37° C., 5% CO2) to allow for cell attachment. Afterwards, 950 μL of complete media were added, yielding a final cell concentration of 5.10+ cells/mL. Media was refreshed every two days.

After 7 days of cell culture, osteoinductive media was added to the cell cultures comprising DMEM, 10% FBS, 1% penicillin/streptomycin, and 0.5% amphotericin, supplemented with 10 mM beta-glycerophosphate, 100 nM dexamethasone, and 80 μM ascorbic acid (Merck, KGaA, Darmstadt, Germany). Cell cultures were maintained for 14 days. After 1, 7 and 14 days, scaffolds were rinsed in PBS, transferred to new wells, and lysed. The cell cultures were lysed with 400 μL of lysis buffer (CellLytic, Sigma Aldrich, Sweden) and shaken for 15 min at 300 rpm. Cell lysates were collected and frozen at −20° C. until use.

Lactate dehydrogenase enzymatic assay (LDH, TOX7, Merck, KGaA, Darmstadt, Germany) was used to quantify cell adhesion and proliferation from the cell lysates at 1, 7, and 14 days, according to the manufacturer's protocol. Absorbance was measured at 690 nm-490 nm in a spectrophotometer (Multiscan Ascent, Thermofisher Scientific Inc., Waltham, MA). LDH values were normalized to control samples comprising empty wells with cells at each corresponding time point, and expressed as x-fold change. Cell differentiation was investigated by alkaline phosphatase (ALP) expression in the cell lysates at 1, 7, and 14 days. ALP substrate (Merck, KGaA, Darmstadt, Germany) was used for the quantification using a standard calibration of p-nitrophenol dilutions (Sigma Aldrich, Sweden), and the absorbance was measured at 405 nm. ALP values were normalized by the total protein content in the cell lysates measured by bicinchoninic acid assay (Pierce™ BCA Protein Assay Kit, ThermoScientific, Rockford, IL) at 540 nm. After normalization by the protein content, ALP values were normalized by each corresponding time control sample and expressed as x-fold change. Three technical replicas were used per experiment and two biological replicas were performed.

### 1.4.2 Osteoblast Morphology

Fluorescence imaging was performed of scaffolds and control cover slips seeded with cells at 1 and 14 days to assess cell morphology by immunochemistry. The cells were fixed with 4% v/v paraformaldehyde for 15 min at room temperature, rinsed with PBS (×3) and permeabilized using 0.1% Triton X-100 (Merck, KGaA, Darmstadt, Germany) at room temperature for 20 min. After rinsing with PBS (×3), cell cytosol was stained with carboxyfluorescein diacetate (500 nM, 400 μL CFDA, Merck, KGaA, Darmstadt, Germany) for 15 min at room temperature in the dark, and cell nuclei were stained with 4,6-diamidino-2-phenylindole (300 nM, 400 μL DAPI, Invitrogen, Massachusetts, USA) at room temperature in the dark for 1 h. The scaffolds were rinsed with PBS (×3) and observed using a Leica microscope (Leica Dmi8, Mycrosystems CMS GmbH, Wetzlar, Germany). One sample per time point, and one control sample consisting of glass cover slips were used for imaging.

### 1.4.3 Primary Human Osteoblasts Studies

### 1.4.3.1 Isolation Description

Primary human osteoblasts (hOB) were isolated from human femoral heads after assessment by the Swedish Ethical Review Authority (approval number: 2020-04462) following a previously published protocol. Briefly, bone parts were diced into small fragments of 1-2 mm, rinsed with PBS and cell culture media, and placed in 25 cm² flasks containing alpha modified minimum essential medium (α-MEM, Merck, KGaA, Darmstadt, Germany), 10% FBS, 1% penicillin/streptomycin and 0.5% amphotericin. Media was refreshed weekly. Cells were expanded in 75 cm² until passage 4 to 6, and subsequently used for the experiments.

### 1.4.3.2 Cell Proliferation and Differentiation

Cell proliferation and differentiation on the three Ti alloys were evaluated at 1, 7, 14 and 28 days. Prior to seeding, samples were preconditioned with complete media comprising α-MEM, 10% FBS, 1% penicillin/streptomycin and 0.5% amphotericin overnight. Media were removed and 10⁶ hOB were seeded per scaffold in 50 μL droplets and placed in the incubator for 1 h to allow for cell adhesion. Afterwards, 950 μL of complete media were added, yielding a final cell concentration of 5·10⁴ cells/mL. Control samples consisting of empty wells (wells without scaffolds) were used. 950 μL of complete media were added to the control samples immediately after cell seeding to avoid drying in the incubator.

Cell cultures were sustained up to 28 days, and media was refreshed every 2 days. After 7 days of cell culture, osteoinductive media comprising complete media supplemented with 10 mM betaglycerophosphate, 100 nM dexamethasone, and 80 μM ascorbic acid (Merck, KGaA, Darmstadt, Germany) was used. All experiments were performed using three technical replicas for each sample, and three biological replicas using different patient-derived hOB. At each specific time point (1, 7, 14 and 28 days) seeded samples were rinsed with PBS, and transferred to new plates for lysis. 400 μL of lysis buffer (CellLytic, Sigma Aldrich, Sweden) were added and shaken for 15 min at 300 rpm. Cell lysates were collected and frozen at −20° C. until use. LDH, ALP and BCA were measured from the cell lysates at 1, 7, 14, and 28 days, as described herein in 1.4.1.

### 1.4.3.3 Mineralization

Mineral deposits were investigated using Alizarin Red (AR, Merck, KGaA, Darmstadt, Germany) staining. After 14 and 28 days of cell culture, cells on the scaffolds were fixed using 70% ice cold ethanol at room temperature for 1 h. Subsequently, the scaffolds were rinsed with distilled water and stained with AR (40 mM, Merck, KGaA, Darsmtadt, Germany) at pH 4.2 at room temperature for 10 min. Next, the scaffolds were rinsed with distilled water (×5), followed by PBS rinsing for 15 min in a shaker to remove any unspecific staining. AR staining was quantified using 10% wt. cetylpyridinium chloride (CPC, Merck, KGaA, Darmstadt, Germany) in 10 mM sodium phosphate solution for 20 min in a shaker at room temperature to decolorize the scaffolds. The supernatants were collected and their absorbance measured at 562 nm. Samples comprising glass cover slips were used as controls. Two samples of each type were used for each experiment, and the experiment was repeated twice using independent patients' cells. To avoid any possible background signal from the scaffolds, samples comprising the same Ti alloys (n=2) without cells were also AR stained, measured and subtracted from the scaffolds seeded with cells.

### 1.4.3.4 Gene Expression

The deoxyribonucleic acid (DNA) levels of osteogenic-related genes in hOB cultured on the three titanium scaffolds were analyzed through real-time quantitative polymerase chain reaction (RT-qPCR) at 3, 7, 14 and 28 days. hOB were cultured as discussed in 1.4.3.2, and lysed at each time point using 400 μL TRIzol™ (Invitrogen, Massachusetts, USA), and stored at −20° C. until used. Ribonucleic acid (RNA) extraction was performed according to the manufacturer's protocol, and the total RNA yield was determined using a nanodrop (ND-1000 Spectrophotometer, Thermofisher Scientific Inc., Waltham, MA). Subsequently, RNA was transformed into complementary DNA (cDNA) by reverse transcription (High Capacity RNA to c-DNA kit, ThermoFisher Scientific Inc., Waltham, MA), and osteogenic-related genes were measured by RT-qPCR (7500 Fast RT PCR System, Applied Biosystems™, Thermofisher Scientific Inc., Waltham, MA).

Runt-related transcription factor 2 (Runx2), collagen type I alpha 1 and 2, (COL1A1, COL2A2), alkaline phosphatase (ALP) and osteocalcin (OCN) gene expression by hOB was quantified by RT-qPCR. The primers listed in Table 1 herein were used for the quantifications, and β-actin was used as house-keeping gene, all purchased from Thermofisher Scientific (Taqman). The CT values from the melting curves were calculated and expressed using the 2ΔΔCT method. Triplicate samples for each time point were used for the gene expression using one patient hOB.

TABLE 1
Taqman probes for the primers used
in gene expression quantification.

| Acronym | Name | Taqman assay number ID |
|---|---|---|
| AGTB | β-actin | 4333762T |
| Runx2 | Runt related transcription factor 2 | Hs00231692_m1 |
| COL1A1 | Collagen type I alpha 1 | Hs00164004_m1 |
| COL1A2 | Collagen type I alpha 2 | Hs01028970_m1 |
| ALPL | Alkaline phosphatase | Hs01029144_m1 |
| BGLA | Osteocalcin | Hs01587814_g1 |

### 1.4.3.5 Human OB Cell Morphology

Fluorescent imaging was performed on the scaffolds with cells at 1, 7, 14, and 28 days to assess cell morphology by immunochemistry. The cells were fixed with 4% v/v paraformaldehyde for 15 min at room temperature, rinsed with PBS (×3) and permeabilized using 0.1% Triton X-100 (Merck, KGaA, Darmstadt, Germany) at room temperature for 20 min. After rinsing with PBS (×3), cell cytosol was stained with CFDA (500 nM, 400 μL CFDA, Merck, KGaA, Darmstadt, Germany) for 15 min at room temperature in the dark, and cell nuclei were stained with DAPI (300 nM, 400 μL DAPI, Invitrogen, Massachusetts, USA) at room temperature in the dark for 1 h. The scaffolds were rinsed with PBS (×3) and observed using a Leica microscope (Leica Dmi8, Microsystems CMS GmbH, Wetzlar, Germany).

One sample per scaffold and per time point was used, and controls comprising glass cover slips were included. Osteocalcin (OCN) staining was included for hOB immunochemistry. After 14 and 28 days, cells on the scaffolds were fixed using 4% v/v paraformaldehyde for 20 min at room temperature, rinsed with PBS (×3) and permeabilized using 0.5% Triton X-100 (Merck, KGaA, Darmstadt, Germany) at room temperature for 15 min after rinsing with PBS (×3). The cell cytosol was stained using CFDA as described in 1.4.2. Afterwards, the samples were blocked with normal 10% goat serum (s-1000, Sigma Aldrich, Sweden) solution prepared in PBS containing 2% bovine serum albumin (BSA) and 0.3% Triton X-100 for 30 min. OCN antibody (20 μg/mL mouse anti-human/rat OCN, MAB1419, R&D Systems, United Kingdom) solution in PBS/2% BSA/0.3% Triton X-100 was added and incubated overnight at 4° C. Afterwards, the samples were rinsed in PBS/1% Triton X-100 (×4), and the secondary antibody (1:200, goat anti-mouse, Biotin Novus NB7537, United Kingdom) was added and incubated for 30 min under agitation at room temperature. The samples were rinsed with PBS/1% Triton X-100 (×4), and then stained with Dylight red (20 μg/mL, Thermofisher Scientific Inc., Waltham, MA) and DAPI dissolved in PBS for 30 min at room temperature, followed by rinsing with PBS/1% Triton X-100 (×4), and finally observed using a Leica microscope (Leica Dmi8, Microsystems CMS GmbH, Wetzlar, Germany).

One sample per scaffold and per time point was used, and controls comprising glass cover slips were included. Additionally, fixed cells on scaffolds were observed by FE-SEM after 1, 7, 14 and 28 days. After cell fixation, the scaffolds were dehydrated following increasing ethanol series (10-30-50-70-90-99% ethanol) for 15 min each, and dried. A relatively thin layer of 5-8 nm gold palladium was sputtered prior to imaging. One scaffold per sample type and time point was included for FE-SEM imaging.

### 1.4.3.6 Silver Release and Effects on Human OB

The silver release from silver coated samples was quantified by inductively coupled plasma optical emission spectroscopy (ICP-OES, Avio 200, Perkin Elmer, USA) using an Argon flow of 8 L/min, and a pump rate of 1 mL/min. The supernatants from hOB cell cultures were collected and the total silver release was analyzed. The scaffolds were hence incubated with 1 mL of complete cell culture media comprising of α-MEM, 10% FBS, 1% penicillin/streptomycin and 0.5% amphotericin at 37° C. over 28 days. The media were replaced every two days, and supernatants were used to quantify the silver release at 1, 2, 3, 5, 7, 9, 14, 19, 21, 26 and 28 days. Triplicate supernatants were used from 3 independent samples (n=3), diluted using 2% nitric acid (HNO3, Merck, Germany), and using cell culture media as blank controls.

The in situ silver effects on hOB were investigated by exposure to silver supplemented medium. hOB were isolated as described in 1.4.3.1 and cultured in α-MEM, 10% FBS, 1% penicillin/streptomycin and 0.5% amphotericin. Cells at passage 4 were seeded in 24-well plates (35.000 cells/mL) for 24 h. Afterwards, cell culture medium supplemented with 0, 5, 10 or 20 ppm AgNO3 (Sigma-Aldrich) was added to the cells. The silver medium was refreshed every two days, and the cells were treated for five days (120 h), during which cell morphology, fission and mobility were examined using live-image microscopy (Leica DMi8 Microscope with INCUBATORi8 environmental chamber) over 20 h. A microscopic picture (10× magnification; exposure time 10 ms) was taken every 15 min, and exported as a time-lapse video (5 frames/s).

### 1.5 Statistics

Antimicrobial properties are presented as mean±standard error, together with median values (squares). Triplicate samples were used for each biological experiment, and four biological replicas were performed for adhesion, and six replicas for the biofilm formation studies. Cell studies on proliferation, differentiation and gene expression are presented as mean±standard error. Three technical replicas were used for each study. Cell proliferation and differentiation for both cell lines was assessed through two biological replicas, and primary cells, through three biological replicas, using three different patient's cells in the case of hOB. Levene's test was used to ascertain homogeneity of variance, and differences between group means were assessed by one-way ANOVA and Tukey's post-hoc test. The threshold of statistical significance was set at p<0.05.

### 2 Results

### 2.1 Samples Characterization

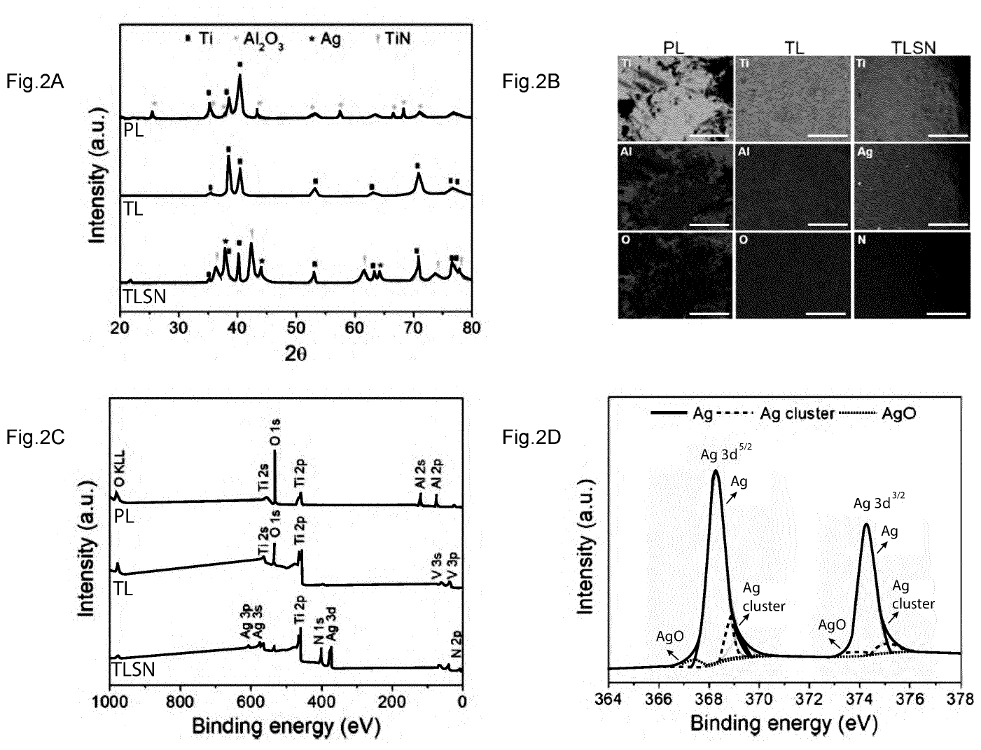

The samples phase composition were measured by XRD, with the results shown in FIG. 2A. The results, as shown in FIG. 2A, indicate a main Ti crystalline phase for dense PL samples with peaks attributed to aluminum oxide (Al2O3). Porous TL samples had a substantially pure Ti phase, while the silver-coated analogues, TLSN, depicted crystalline phases corresponding to silver (Ag) and titanium nitride (TiN).

EDS color mapping images are shown in FIG. 2B, depicting titanium, aluminum, and oxygen on PL and TL samples while silver and nitrogen were seen at the surface level of the silver coated analogues TLSN. The semiquantitative EDS mapping analyses of the sample surfaces yielded a titanium content of 32.5 at. % for PL, and 64.7 at. % for TL. PL samples showed additional values for alloying elements such as aluminum and vanadium of 11.5 at. % and 1.5 at. %, respectively, and 45 at. % of oxygen. TL samples were composed of 8.3 at. % aluminum, 2.7 at. % vanadium, and 16.3 at. % oxygen. The silver-coated TLSN contained 56.5 at. % nitrogen and 7 at. % silver in addition to 33.2 at. % titanium.

Silver coating was substantially homogeneously distributed on the TLSN scaffolds, both at the printed pillars and the pores; the silver content inside the pores slightly diminished compared to the pillars from 7 to 5.4 at. %. Carbon contents below 9 at. % were also found in all samples, ascribed to deposition during measurement from electron bombardment. The chemical surface analyses from XPS results are shown in FIG. 2C. FIG. 2C illustrates the presence of oxygen, aluminum and titanium for PL samples. TL scaffolds include additional peaks for vanadium, and less intense oxygen peaks, while TLSN scaffolds include an increase in the nitrogen signal, and the presence of silver. The total content of silver investigated by XPS was 7.5 at. %. Further analyses on the silver peaks revealed the presence of metallic silver Ag (87 at. %), located at 368.3 eV. Low amounts of oxidized silver (3.8% at.) were observed, corresponding to silver (II) oxide AgO, located at lower binding energies, 367.4 eV. An additional peak was found at 369.2 eV, attributed to silver clusters (9.2 at. %), shown in FIG. 2D.

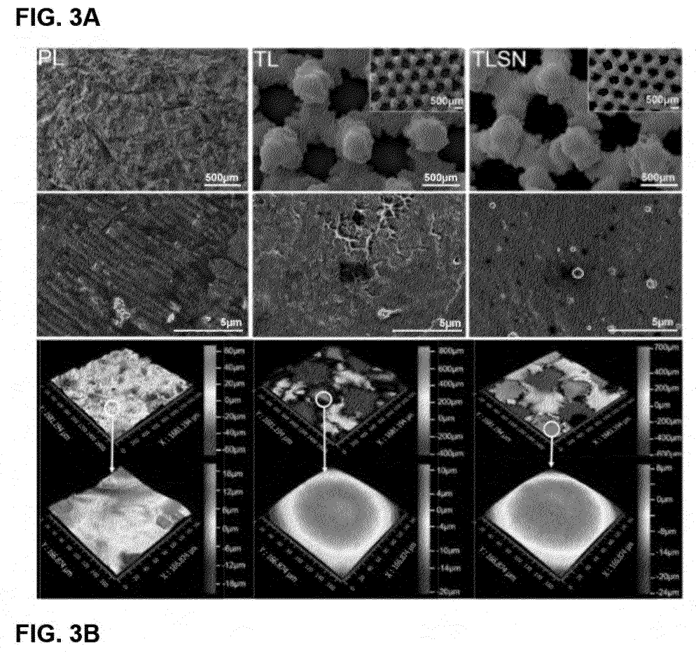

Surface morphology was assessed by FE-SEM, with the resultant images shown in FIG. 3A. As seen in FIG. 3A, the surface of PL samples was relatively rough after grit-blasting, while TL and TLSN had 500-600 μm pores. As also seen in FIG. 3A, the additively manufactured pillars of both porous samples (TL and TLSN, second and third columns respectively) showed smoother surfaces compared to PL samples. The difference in surface roughness was confirmed by WLI measurements where PL samples had a rougher surface compared to TL and TLSN printed parts.

The results of this WLI measurement are illustrated in FIG. 3B. The surface roughness of PL samples were measured within a scanned area of 1.68×1.68 mm², which yielded a Ra of 12.6±2.5 μm. The values for TL and TLSN samples for the same scanned area were 227±60 μm for TL and 237±70 μm for TLSN, depicting the average peak-to-valley distance from the printed structure. To further analyze the surface roughness of the samples, a smaller scanned area of 167×167 μm² was considered, and the Ra, Rsk, and Rku are for each sample are shown in Table 2. The surface roughness values, symmetries, and distributions were similar for both printed pillars (TL and TLSN), while PL roughness had higher values. The cracks and voids observed in PL samples resulted in negative values in Rsk, and higher kurtosis values.

TABLE 2
Roughness values of the three samples expressed as mean ± standard
deviation for a scanned area of 167 × 167 μm².

| Sample type | Rₐ (μm) | Rₛₖ (μm) | Rₖᵤ (μm) |
|---|---|---|---|
| PL | 9.2 ± 3.4 | −0.02 ± 1.1 | 2.5 ± 1.0 |
| TL | 4.7 ± 1.2 | 0.1 ± 1.2 | 1.7 ± 0.2 |
| TLSN | 5.0 ± 1.4 | 0.2 ± 1.2 | 1.9 ± 0.7 |

### 2.2 Antimicrobial Effects

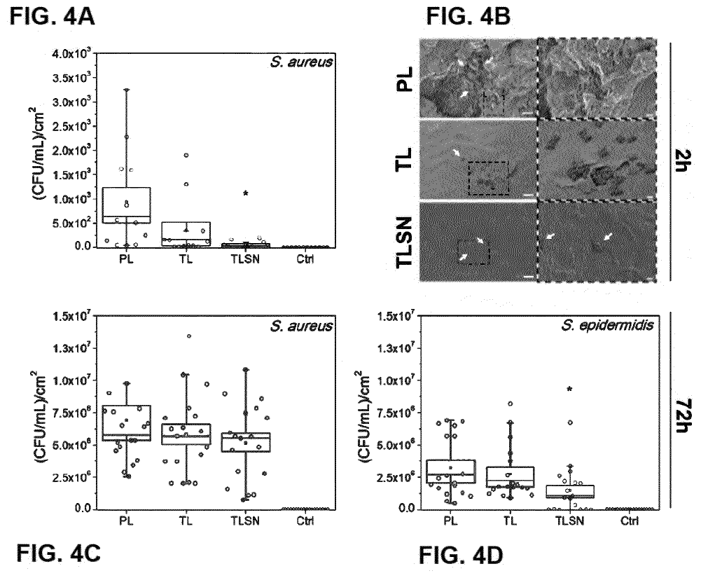

The bacterial adhesion after 2 h of incubation on the three samples was evaluated using S. aureus. The CFU counts were highest on PL dense samples, followed by TL porous analogues, while CFU counts were lowest on silver-coated TLSN, as shown in FIG. 4A. The antibacterial affect of TLSN was further investigated by FE-SEM, indicating the presence of S. aureus aggregates on both PL and TL, while only very few, isolated bacteria were found on TLSN, as shown in the images of FIG. 4B. Bacterial biofilm formation was studied after 72 h of incubation of samples with either S. aureus (results shown in FIG. 4C) or S. epidermidis (results shown in FIG. 4D).

CFU counts thus obtained were four orders of magnitude higher than after 2 h, and smaller differences between samples were observed. CFU counts were similar on PL and TL samples, whereas a small but statistically non-significant decrease was seen on silver-coated TLSN, as shown in FIG. 4C. Biofilm formation by S. epidermidis was attenuated when compared to S. aureus on all samples, as shown in FIG. 4D. However, CFU counts after incubation of TLSN with S. epidermidis were only significantly lower compared to PL.

### 2.3 Cell Studies

### 2.3.1 Cell Lines

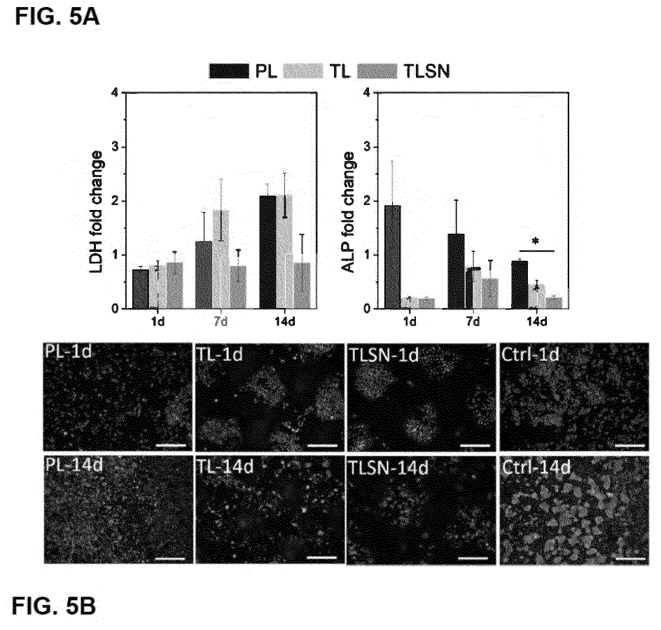

Cell viability was first assessed using SaOs-2 cells at 1, 7 and 14 days after seeding on the investigated samples, indicating similar proliferation rates on PL and TL samples, as illustrated in FIG. 5A. After 7 days of culture, higher cell viability measured by LDH was found on TL samples than on PL samples (1.8 fold change compared to 1.2). In contrast, proliferation of SaOs-2 cells on silver-coated TLSN samples was significantly reduced after 7 and 14 days (0.86 fold change at day 1, 0.80 at day 7, and 0.86 at day 14). SaOS-2 cell differentiation assessed by ALP production indicated a lower degree of differentiation on both porous samples TL and TLSN when compared to PL samples (as illustrated in FIG. 5A), and this difference in ALP concentrations was statistically significant after 14 days. The investigation of cell morphology by immunofluorescence correlated with the above-described enzyme measurements, indicating similar cell coverage of all scaffolds after 1 day of cell culture, while decimated cell populations were observed on silver-coated TLSN samples when compared to TL analogues and PL samples after 14 days, as can be seen in the images of FIG. 5B.

### 2.3.2 Human Osteoblasts

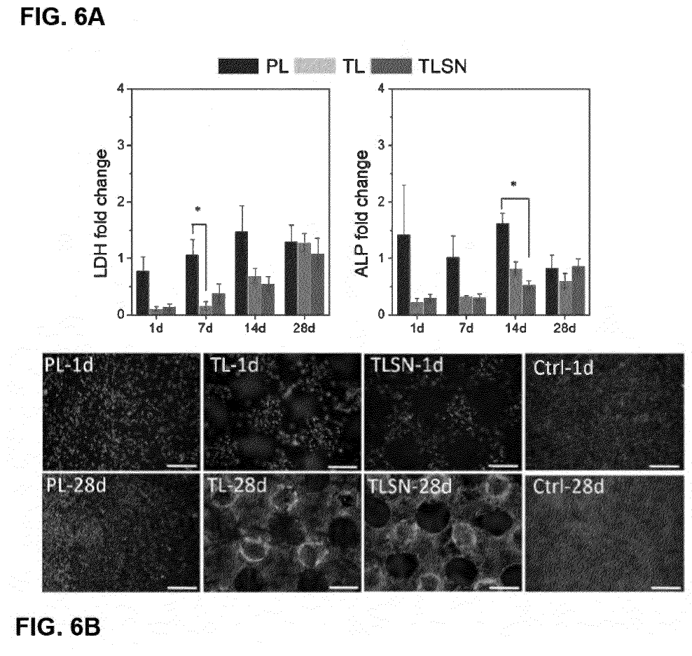

Primary hOB viability on the different scaffolds was evaluated at 1, 7, 14 and 28 days, with the results shown graphically in FIG. 6A. Measurements of LDH indicated lower cell adhesion on porous TL and silver-coated TLSN at 1 day compared to dense PL scaffolds (0.11 and 0.13 fold change for TL and TLSN, respectively, compared to 0.77 fold change in PL). This trend was maintained after 7 days, indicating significantly higher cell proliferation on PL compared to both porous scaffolds.

After the addition of osteoinductive media at 7 days, the cell proliferation rates increased both on dense PL and on porous TL and TLSN. After 28 days of cell culture, cell viability was similar on all samples and higher than on control samples (fold change of 1.3 for PL, 1.3 for TL, and 1.1 for TLSN). The differentiation of hOB measured by ALP production indicated enhanced differentiation on PL scaffolds compared to porous TL and TLSN scaffolds after 7 and 14 days. However, after 28 days of cell culture, ALP expression was upregulated on porous TL and TLSN, reaching similar values to those on PL, with no statistically significant differences between groups.

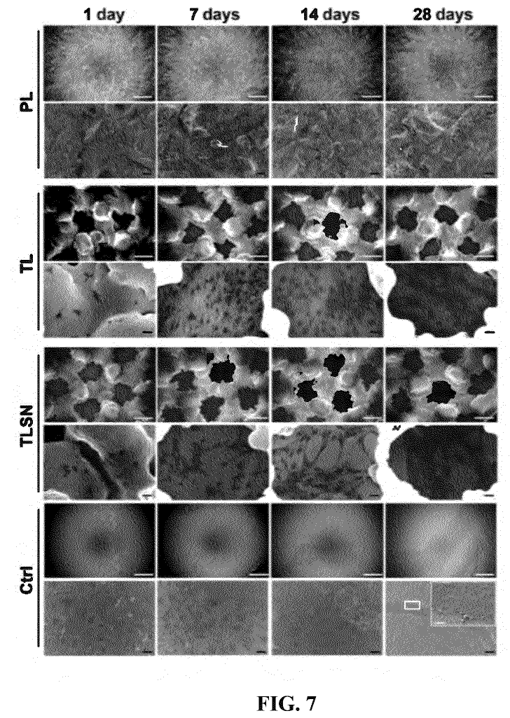

Cell morphology was evaluated by immunostaining of cell nuclei and cytosol, with the results shown in the images of FIG. 6B. The images of FIG. 6B indicate good cellular adhesion after 1 day, evolving into a full coverage of all scaffold surfaces after 28 days for all three types of scaffolds. No gross differences in hOB coverage or morphology were observed when comparing silver-coated TLSN with uncoated TL. Similarly, cell morphology evaluated by FE-SEM at 1, 7, 14 and 28 days confirmed the increasing cellular coverage of all scaffold surfaces over time, with the images resulting from this further evaluation shown in FIG. 7. As can be seen in FIG. 7, a compact cell monolayer was achieved after 28 days, both at the printed protrusions and inside the pores, and with no obvious differences in cell coverage becoming visible when comparing silver-coated TLSN with uncoated TL.

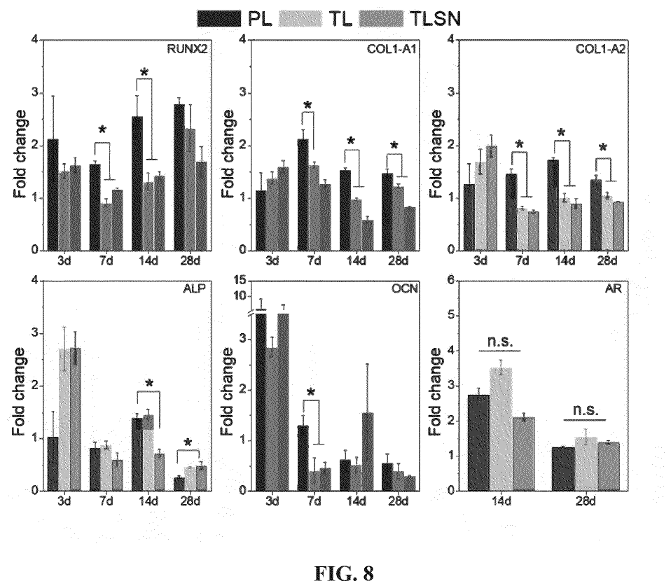

Osteogenic gene expression of RUNX2, COL1A1, COL1A2, ALP and OCN by hOB was evaluated by RT-qPCR after 3, 7, 14 and 28 days, with the results graphically shown in FIG. 8. To generate the data of FIG. 8, all samples were compared to control samples comprising wells seeded with the same cell density but without scaffolds, and measurements were expressed as fold change after normalization to house-keeping gene β-actin. Upregulation of RUNX2 was observed on PL samples regardless of time point, but the upregulation of RUNX2 on TL and TLSN samples was significantly lower compared to PL after both 7 and 14 days. COL-1 expression was significantly higher on dense PL compared to porous TL and TLSN after 7, 14 and 28 days, but no significant differences were found between the two porous scaffolds TL and TLSN.

The expression of ALP and OCN was high on all scaffolds after 3 days. After 7 days of cell culture, the expression of both ALP and OCN was lower than after 3 days on all samples. After 14 days, ALP gene expression was higher on both PL and TL than on silver-coated TLSN, whereas after 28 days ALP expression was higher on porous TL and TLSN than on PL samples. OCN expression was downregulated on all scaffolds after 7, 14 and 28 days compared to the early measurement after 3 days, without statistically significant differences between the three scaffolds at any time point except for 7 days, where porous samples conferred lower OCN expression than dense PL. Additionally, osteogenic gene expression was never statistically significantly lower on silver-coated TLSN when compared to uncoated TL samples.

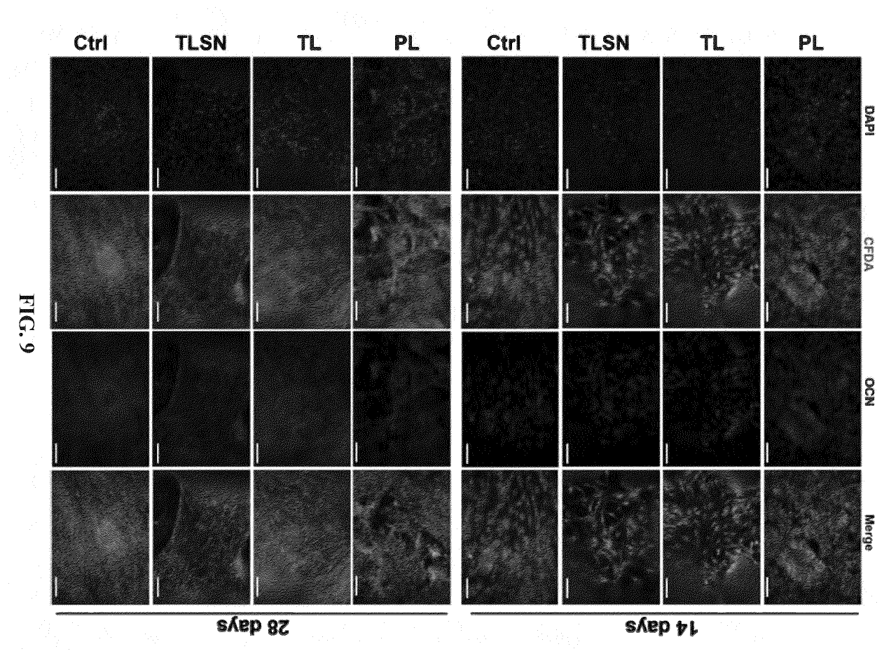

The mineralization of the scaffolds induced by hOB and quantified by AR staining showed mineral deposits on all scaffolds when compared to control samples. Overall, all scaffolds similarly promoted mineralization, with no significant differences between silver-coated TLSN and uncoated TL. The immunofluorescence images, shown in FIG. 9, of hOB demonstrate the cell morphology after 14 and 28 days on all investigated scaffolds. Larger cells were observed on porous TL and TLSN scaffolds, often aligned along the printed bridges connecting pillars, while PL and Ctrl samples showed larger cell aggregates. After 14 days, DAPI staining showed higher numbers of cell nuclei on PL and Ctrl samples but slightly lower on porous TL and TLSN scaffolds. However, after 28 days of culture, numbers of nuclei were similar on PL and TL scaffolds but slightly lower on TLSN.

Cytoskeleton staining with CFDA after 14 days showed that hOB grown on porous TL and TLSN scaffolds appeared larger and slightly more elongated than those on PL. After 28 days, full cell coverage was observed on all scaffolds, with higher numbers of cell nuclei on porous TL, and alignment of cells was observed along the printed struts of porous TL and TLSN. The fluorescence intensity of OCN was higher on PL after 14 days, although after 28 days of cell culture, OCN appeared more intense on porous TL compared to dense PL, reaching similar OCN intensity as that of Ctrl samples. Silver-coated TLSN had the lowest OCN staining intensity, although an increase in OCN signal was observed from 14 to 28 days even on these samples.

### 2.3.3 Silver Release and Effects on Osteoblasts

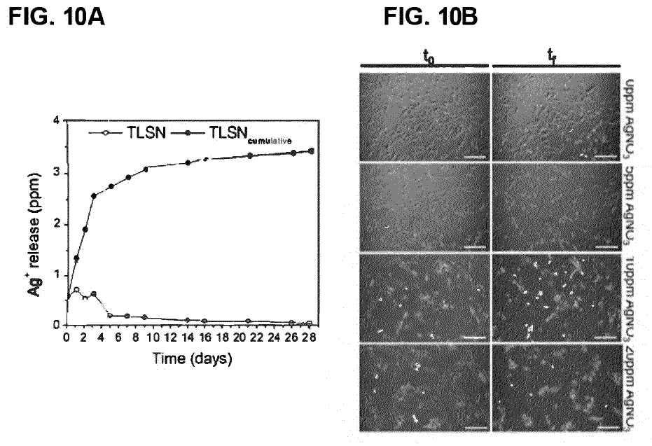

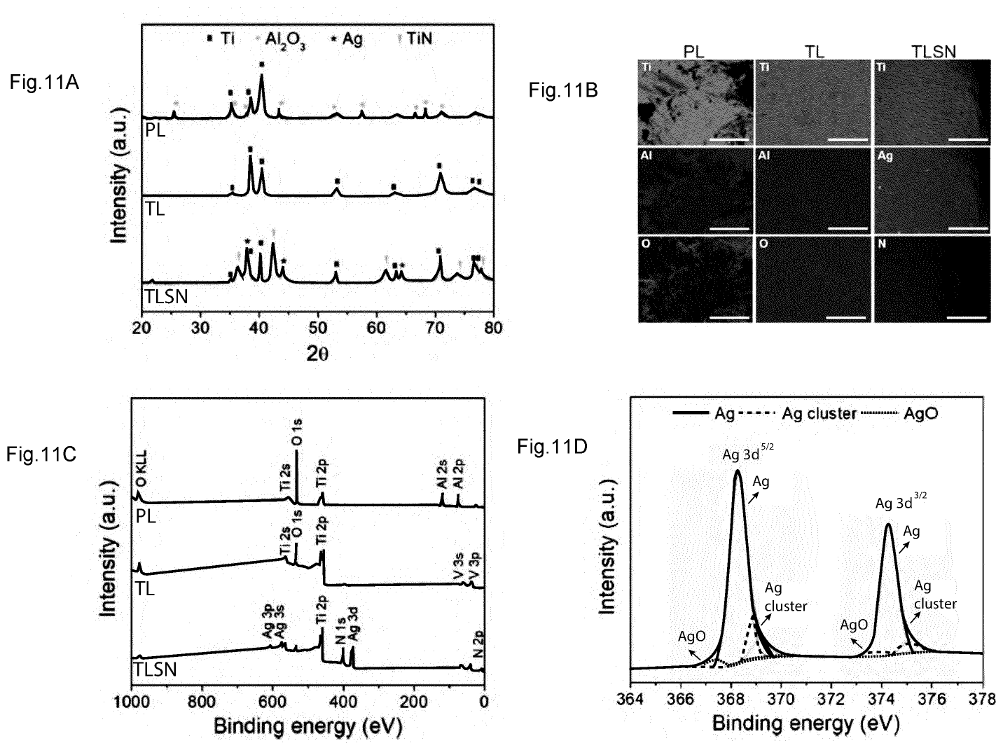

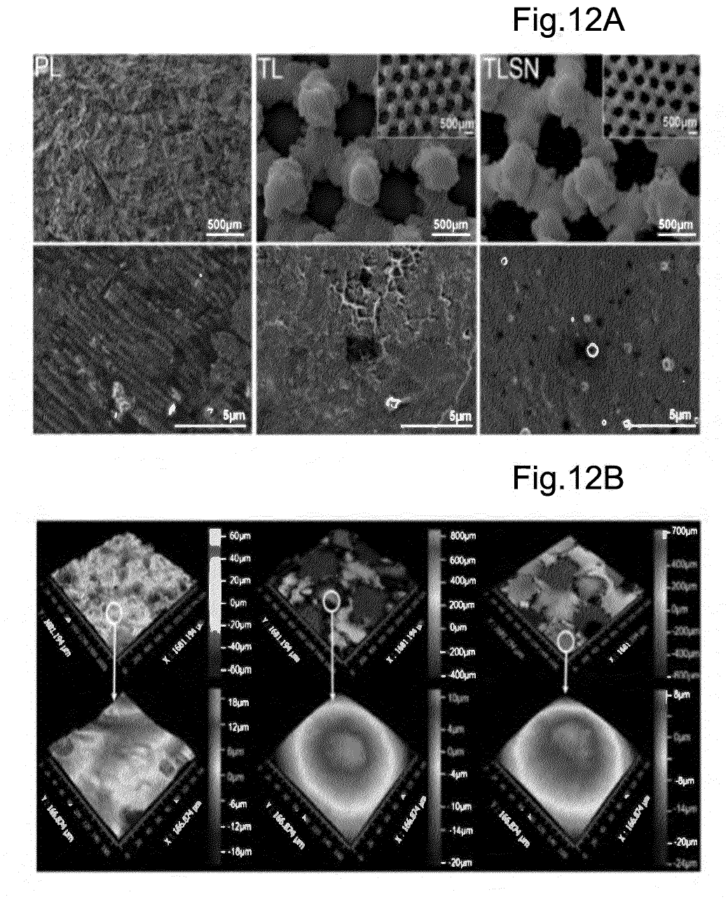

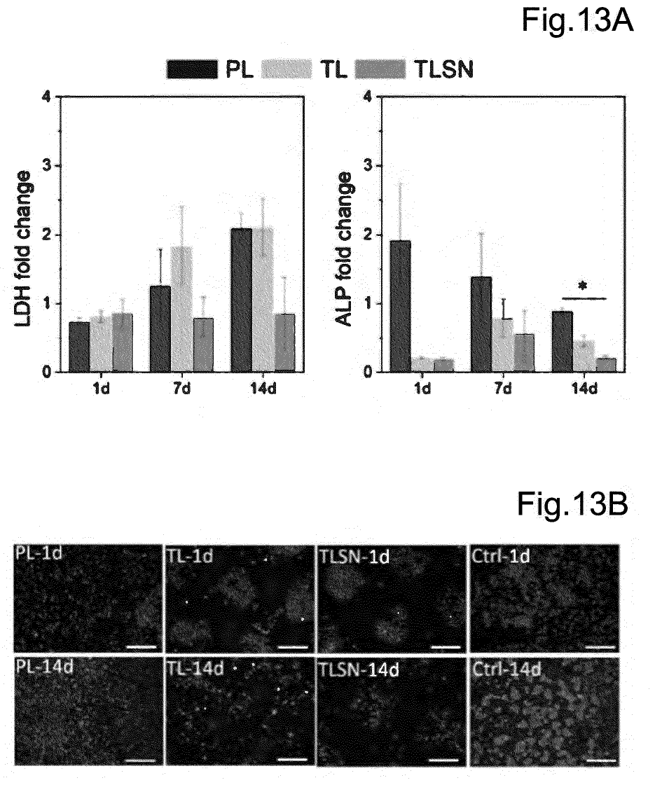

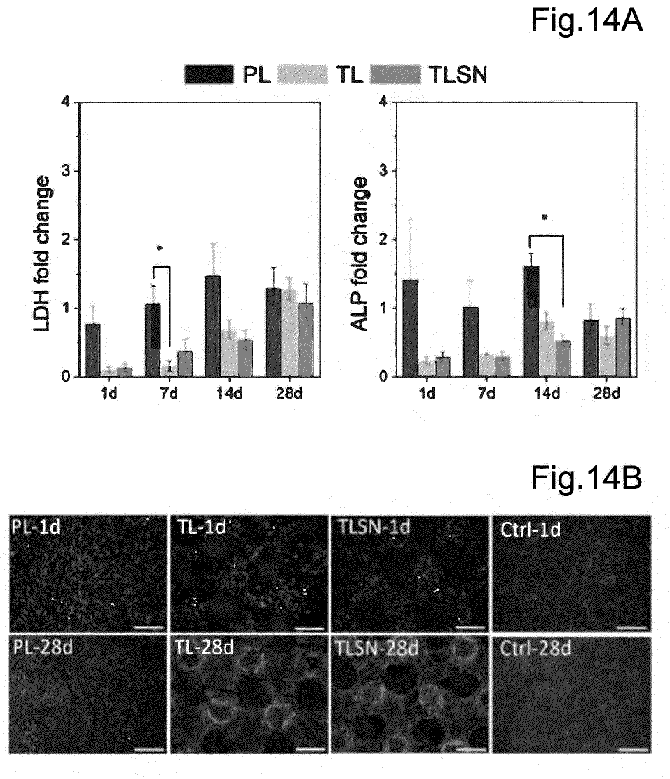

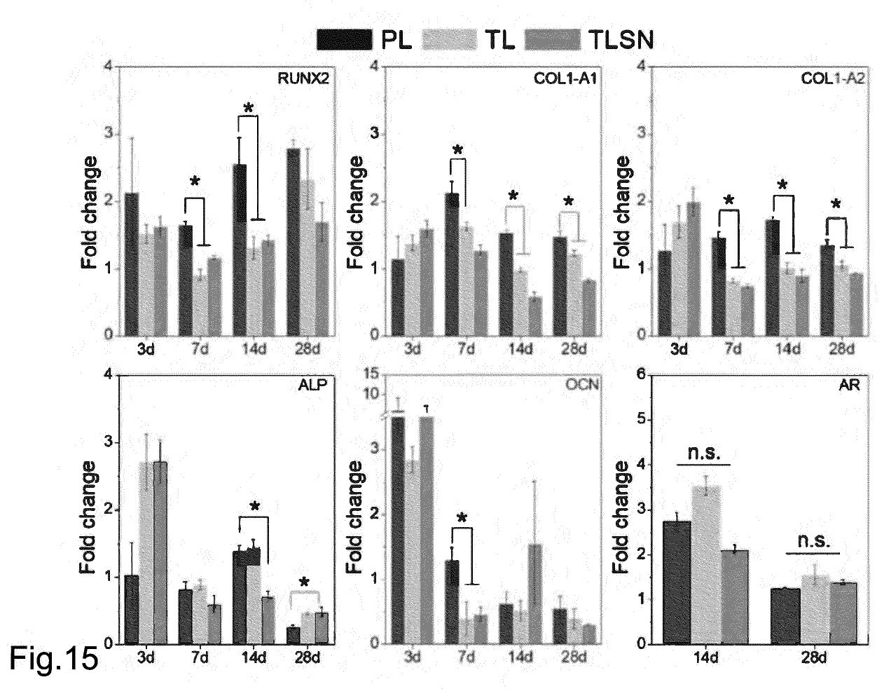

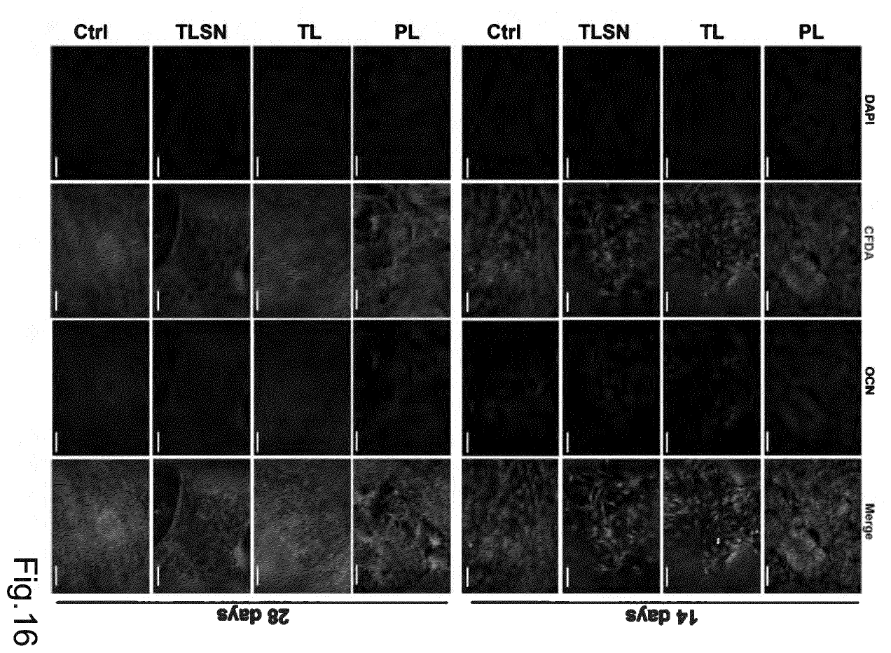

The silver release from cell culture supernatants was analyzed by ICP-OES up to 28 days on silver-coated TLSN, with the gathered data shown graphically in FIG. 10A. A maximum silver release of 0.74 ppm after 1 day was determined. After 5 days, the silver release decreased substantially below 0.2 ppm, and a further decrease below 0.1 ppm was observed after 14 days, with this concentration being maintained up to 28 days. The cumulative silver release for TLSN, also shown graphically in FIG. 10A, showed a plateau after 7 to 9 days, with values below 3.5 ppm.

The effect of silver ions on hOB morphology was assessed by the addition of different silver nitrate concentrations to the cell culture medium over 5 days followed by 20 h time-lapse microscopy, with cell morphology images shown in FIG. 10B. The evaluation of cell morphology demonstrated that concentrations of silver below 5 ppm did not impair cell survival, indicated by the presence of mobile cells during the entire culture time. In contrast, concentrations of 10 and 20 ppm did affect the mobility of cells and induced apoptosis.

### 3. Discussion

Uncemented joint arthroplasty would benefit from improved material properties to achieve both osseointegration and reduced risk of bacterial infection. The use of porous metals has been studied due to elasticity, reduced stress shielding affects and enhanced osseointegration, with resulting improvements to both primary and secondary implant stability. However, with the increased surface area inherent to porous biomaterials the risk of bacterial adhesion is increased, creating a desire for porous biomaterials to be imbued with antimicrobial properties.

Ionic metal coatings such as silver have been used on clinically used arthroplasty implants, but concerns related to the possible cytotoxic effects of silver on surrounding bone cells have, until now, inhibited the use of silver on parts of the implants intended for bone ingrowth. The effects of a silver coating are reviewed herein in conjunction with an additively manufactured Ti6Al4V porous titanium scaffold on bacterial adhesion and biofilm formation along with its biocompatibility.

The chemical structure of the three scaffolds analyzed by XRD and EDS, shown in FIGS. 2A and 2B demonstrate the similarity of the subjacent substrate based on Ti6Al4V alloy, while the sample with a 4.5 μm silver titanium nitride layer coating showed peaks on XRD corresponding to titanium nitride, silver, and the subjacent titanium alloy. In addition, traces of aluminum oxide were found on PL, compatible with the corundum grit-blasting process. These finding were consistent with the XPS chemical analyses shown in FIG. 2C, where O and Al peaks were present for PL samples, masking the presence of the underlying substrate, as no vanadium peaks were found.

On the contrary, TL scaffolds showed peaks for Ti, Al and V, together with O, corresponding to the substrate Ti6Al4V alloy. The total atomic percentage of silver in silver-coated TLSN porous scaffolds was 7% at. when measured by EDS. XPS analyses were in agreement with the silver amount found by EDS. In depth analysis of the silver contents showed that the majority of the silver present in the coating of TLSN corresponded to metallic silver. The PVD process, occurring under vacuum and N2 atmosphere renders the unoxidized state of silver, since the samples were measured ‘fresh’. Upon contact with air, such as in our cell culture studies or implanted, silver becomes oxidized which results in a continuous release of small amounts of ions, yielding antibacterial effects. A small percentage of oxidized silver below 4 at. % was observed, and shown in FIG. 2D, to which the antibacterial effects of TLSN are also ascribed.

The morphology of the samples varied according to the manufacturing process, with PL having a rough surface from the grit-blasting procedure with a surface roughness around 10 μm, as shown in Table 2. In contrast, TL and TLSN had highly porous structures with pore sizes ranging between 500 and 600 μm, a peak-to-valley distance of about 250 μm for the 3D printed structure, and a surface roughness of about 5 μm for the printed pillars for both porous scaffolds, as seen in Table 2 and in FIG. 3B.

TL, with its slightly smoother surface and less skewed roughness yielded less bacterial adhesion at 2 h compared to PL. Despite that micro-sized patterned features have been demonstrated to modify the adhesion and arrangement of bacteria, the differences in surface chemistry between PL and TL could also play a role in S. aureus adhesion. The initial adhesion value for S. aureus was reduced by 63% on TL samples compared to PL, while an even stronger and statistically significant 94.5% reduction on TLSN samples compared to PL was found, the latter observation being attributable to the antibacterial effects of the silver coating.

The absolute effect of silver in bacterial adhesion can be quantified by the reduction in adhesion when comparing TL and TLSN samples; a reduction of 85% was observed. These results were further supported by SEM, where PL was covered by larger aggregates of bacteria, followed by TL, while TLSN was only populated by few, isolated bacteria, as seen in the images of FIG. 4B.

Long-term exposure of silver-coated TLSN to S. aureus, such the 72 h biofilm formation assessment, resulted in no significant reduction in CFU counts, as seen in FIG. 4C. Although TL and TLSN both induced a reduction in the number of CFU by 15 and 25% compared to PL, the differences were not statistically significant. In comparison, biofilm formation by Staphylococcus epidermidis was more susceptible to silver, since the presence of silver resulted in a reduction of 46% in CFU count when comparing the two porous scaffolds TL and TLSN with each other, as seen in FIG. 4D. Given the increase in surface area in porous samples, and its consequences in increasing bacterial adhesion, silver coating on porous TLSN is effective in reducing initial S. aureus adhesion.

One aspect in the use of silver coating is to prevent cytotoxic effects to mammalian cells. Current clinical data using silver-coated prosthetics is confined to approaches where the implant parts that are in direct contact with bone are exempt of silver coating. In contrast, in the present disclosure, the biological assessment with hOB showed no detrimental effects of silver on mammalian cells, as illustrated in FIGS. 6-9. Silver release of the disclose device measured over 28 days showed a maximum cumulative silver release below 3.5 ppm, as shown in FIG. 10A. The time-lapse microscopy of cell cultures exposed to silver nitrate, as shown in FIG. 10B, demonstrate that cell mobility and morphology were affected at silver concentrations of 10 and 20 ppm, while for silver concentrations up to and including 5 ppm, no morphological sign of toxicity to hOB was observed. Also, when analyzing the metabolic activity of hOB in contact with silver, no detrimental effects in cell survival, proliferation and differentiation were observed, as shown in FIG. 6A. Although cell viability was lower at earlier time points (1 and 7 days) on porous samples than on dense analogues, both porous TL and TLSN yielded similar values over 28 days of culture, and differentiation measured by ALP reached similar values to those of dense PL after 28 days. Despite the lower cell counts at 1 day, cells proliferated and fully covered scaffolds' surface by a confluent layer after 7 days, as shown in FIG. 7.

Analysis of cell metabolic activity of gene expression by PCR showed that porous samples TL and TLSN, although depicting lower upregulation levels than dense PL, still upregulated matrix deposition and mineralization, as seen in FIG. 8. At earlier time points (3 days), porous samples, TL and TLSN, resulted in higher upregulation of COL1A1, COL1A2, and ALP compared to dense PL. After 7, 14 and 28 days, PL upregulation of osteogenic related genes was higher than porous TL and TLSN, although the later were still higher than control samples (>1 fold change). Despite late markers such as OCN were not upregulated, fluorescence staining showed strong signal for OCN in both TL and TLSN samples, which further increased from 14 to 28 days. The presence of silver coating of TLSN samples had no detrimental effect on cell metabolism of human OB.

The porous silver-coated TLSN investigated here supported proliferation and differentiation of primary human osteoblasts, and upregulated cell matrix gene expression and mineralization. The silver coating of these porous titanium structures simultaneously inhibited initial bacterial adhesion and biofilm formation by the more slow-growing S. epidermidis strain, whereas longer incubation periods still allowed for biofilm formation of the fast-growing S. aureus.

The described embodiments and examples of the present disclosure are intended to be illustrative rather than restrictive, and are not intended to represent every embodiment or example of the present disclosure. While the fundamental novel features of the disclosure as applied to various specific embodiments thereof have been shown, described and pointed out, it will also be understood that various omissions, substitutions and changes in the form and details of the devices illustrated and in their operation, may be made by those skilled in the art without departing from the spirit of the disclosure. For example, it is expressly intended that all combinations of those elements and/or method steps which perform substantially the same function in substantially the same way to achieve the same results are within the scope of the disclosure. Moreover, it should be recognized that structures and/or elements and/or method steps shown and/or described in connection with any disclosed form or embodiment of the disclosure may be incorporated in any other disclosed or described or suggested form or embodiment as a general matter of design choice. Further, various modifications and variations can be made without departing from the spirit or scope of the disclosure as set forth in the following claims both literally and in equivalents recognized in law.

1. A device comprising:
a porous, titanium alloy structure; and
a coating on at least a portion of the structure,
wherein the titanium alloy structure comprises a titanium structural component,
wherein the coating comprises a titanium coating component, a silver coating component, and a nitrogen coating component.

2. The device of claim 1, wherein the silver coating component comprises silver agglomerates.

3. The structure of claim 1, wherein the titanium coating component and the nitrogen coating component comprise titanium nitride.

4. The structure of claim 3, wherein the silver coating component comprises silver agglomerates.

5. The structure of claim 4, wherein a first portion of the silver agglomerates are partially within the titanium nitride and a second portion of the silver agglomerates are within the titanium nitride.

6. The structure of claim 1, wherein the titanium alloy structure further comprises at least one of an aluminum structural component and a vanadium structural component.

7. The structure of claim 6, wherein the titanium alloy structure comprises Ti-6Al-4V.

8. The device of claim 1, wherein the coating is about 3 μm to about 6 μm in thickness.

9. The device of claim 1, wherein the coating comprises about 35% at % titanium structural component, about 10 at % Silver coating component and about 55% at % nitrogen coating component.

10. The structure of claim 1, further comprising a Zr coating between the titanium alloy structure and the silver comprising coating.

11. A device comprising:
a structure configured to contact a portion of a mammal's bone; and
a coating on at least a portion of the structure,
wherein the coating comprises a titanium coating component, a silver coating component, and a nitrogen coating component.

12. The device of claim 11, wherein the silver coating component comprises silver agglomerates.

13. The structure of claim 11, wherein the titanium coating component and the nitrogen coating component comprise titanium nitride.

14. The structure of claim 13, wherein the silver coating component comprises silver agglomerates.

15. The structure of claim 14, wherein a first portion of the silver agglomerates are partially within the titanium nitride and a second portion of the silver agglomerates are within the titanium nitride.

16. A method of inhibiting bacterial adhesion on an implantable apparatus, the method comprising:
inserting the implantable apparatus into a portion of a mammal, to contact a portion of the mammal's bone,
wherein the implantable apparatus comprises any suitable structure capable of insertion into a portion of a mammal and a coating on at least a portion of the structure, wherein the coating comprises a titanium coating component, a silver coating component, and a nitrogen coating component.

17. The method of claim 16, wherein the silver coating component comprises silver agglomerates.

18. The method of claim 16, wherein the titanium coating component and the nitrogen coating component comprise titanium nitride.

19. The method of claim 18, wherein the silver coating component comprises silver agglomerates.

20. The method of claim 19, wherein a first portion of the silver agglomerates are partially within the titanium nitride and a second portion of the silver agglomerates are within the titanium nitride.
